(19) United States
(12) Patent Application Publication
(10) Pub. No.: US 2026/0113180 A1
(43) Pub. Date: Apr. 23, 2026

(54) SECURE COMMUNICATIONS SESSION BETWEEN A MEDICAL DEVICE AND AN EXTERNAL DEVICE

(71) Applicant: Medtronic, Inc., Minneapolis, MN (US)

(72) Inventors: Bo Zhang, Shoreview, MN (US); Thomas J. August, Minneapolis, MN (US); John Louis Clark, Champlin, MN (US); Christopher T. House, Rosemount, MN (US); Robert D. Musto, Champlin, MN (US); John C. Stroebel, Blaine, MN (US); Joel B. Artmann, Elk River, MN (US)

(21) Appl. No.: 19/121,737

(22) PCT Filed: Oct. 18, 2023
(86) PCT No.: PCT/US2023/077190

§ 371 Date: Apr. 16, 2025

(51) Int. Cl. H04L 9/08; G06F 21/60; G16H 40/40; G16H 40/67; H04W 12/50

(52) U.S. Cl. CPC H04L 9/0822; G06F 21/606; G16H 40/67; G16H 40/40; H04L 2209/80; H04L 2209/88; H04W 12/50

(57) ABSTRACT

This disclosure is directed to devices, systems, and techniques for a medical device to securely communicate with an external device. The medical device may establish a secure communications channel over a wireless connection with an external device. The medical device may negotiate with the external device via the secure communications channel one or more encryption keys. The medical device may encrypt communications via the wireless communication with the external device using the one or more encryption keys.

This application is an international application with provisional priority of U.S. Provisional Patent Application No. 63/380,157 , filed 19 Oct. 2022, the entire contents of which is incorporated herein by reference.

### TECHNICAL FIELD

The disclosure relates to device communication between two or more devices.

### BACKGROUND

A computing device may be configured to receive communications from an implantable medical device (IMD). IMDs may be surgically implanted in a patient to monitor one or more physiological parameters of the patient and/or deliver therapy to suppress one or more symptoms of the patient. For example, an IMD may include a cardiac monitor, be configured to deliver cardiac pacing or another electrical therapy to the patient, and/or be configured to terminate tachyarrhythmia by delivery of high energy shocks. A clinician or patient may use an external device to retrieve information collected by the IMD and/or to configure or adjust one or more parameters of the monitoring and/or therapy provided by the IMD.

### SUMMARY

In general, the disclosure is directed to devices, systems, and techniques for an implantable medical device (IMD) implanted in a patient to securely communicate information with an external device. The IMD may collect and/or generate sensitive physiological and/or medical information regarding the IMD and/or regarding a patient in which the IMD is implanted. The IMD may send such information to an external device, such as a medical device programmer or any other computing device, that is used by the patient or a medical care provider to assess the current and historical physiological state of a patient to identify and/or predict impending events or conditions, or that otherwise receives information from the IMD. Given the sensitive nature of the information collected and/or generated by the IMD, the IMD may employ certain techniques to securely communicate with an external device, in order to prevent unauthorized parties from accessing such sensitive information collected and/or generated by the IMD.

An IMD connects to an external device via a wireless connection. In some examples, a wireless connection is established between the external device and the IMD using a Bluetooth Low Energy (BLE) wireless protocol. A BLE wireless protocol is a non-proprietary communication protocol that can reduce cost by reducing or eliminating the need for expensive, proprietary instrumentation. However, a BLE wireless protocol may not provide a secure mechanism for an IMD to communicate with an external device to send and receive sensitive physiological and/or medical information.

In accordance with aspects of the present disclosure, an IMD may securely communicate with an external device to send and receive sensitive physiological and/or medical information by using one or more encryption keys to encrypt information that is communicated between the IMD and the external device via a wireless connection. In order for the IMD and the external device to share the one or more encryption keys, the IMD may establish a secure tunnel over the wireless connection, such as in the form of a Transport Layer Security (TLS) tunnel over the wireless connection, and the IMD may negotiate the one or more encryption keys with the external device via the secure tunnel. Once the IMD and the external device have negotiated the one or more encryption keys, the IMD and the external device may securely communicate sensitive physiological and/or medical information by encrypting communications over a link layer and/or an application layer of the wireless connection using the one or more encryption keys. In this way, an IMD may securely communicate with an external device to send and receive sensitive physiological and/or medical information.

The techniques of this disclosure may provide one or more advantages. For example, by establishing secure tunnel over the wireless connection, the IMD and the external device may be able to securely share, exchange, or otherwise negotiate the encryption keys used to securely communicate over the link layer and/or the application layer of the wireless connection without having to use an out-of-band connection, such as an inductive coupling connection between the IMD and the external device, to negotiate the encryption keys. Not having to use an out-of-band connection to negotiate the encryption keys may reduce the complexity of the IMD and the external device and/or may reduce the manufacturing and/or componentry costs of the IMD because the IMD may not have to be designed to support such out-of-band connections. Furthermore, not having to use an out-of-band connection to negotiate the encryption keys may increase the reliability of the IMD, as the IMD may be able to use a single wireless connection rather than multiple different wireless connections, with potential multiple points of failure, to exchange encryption keys.

In some aspects, a method includes establishing, by processing circuitry of a medical device, a secure communications channel over a wireless connection with an external device; negotiating, by the processing circuitry with the external device via the secure communications channel, one or more encryption keys for secure communication between the medical device and the external device; and encrypting, by the processing circuitry, communications with the external device using the one or more encryption keys.

In some aspects, a medical device is configured for wireless communication, wherein the medical device includes: a memory; communication circuitry configured for wireless communication; and processing circuitry electrically coupled to the communication circuitry and the memory, wherein the processing circuitry is configured to: establish, a secure communications channel over a wireless connection via the communication circuitry with an external device; negotiate, with the external device via the secure communications channel, one or more encryption keys for secure communication between the medical device and the external device; and encrypt communications with the external device using the one or more encryption keys.

In some aspects, an apparatus includes: means for establishing a secure communications channel over a wireless connection with an external device; means for negotiating, with the external device via the secure communications channel, one or more encryption keys for secure communication between a medical device and the external device; and means for encrypting communications with the external device using the one or more encryption keys.

In some aspects, a non-transitory computer-readable storage medium comprising program instructions that, when executed by processing circuitry of a medical device, cause the processing circuitry to: establish, a secure communications channel over a wireless connection with an external device; negotiate, with the external device via the secure communications channel, one or more encryption keys for secure communication between the medical device and the external device; and encrypt communications via the wireless connection with the external device using the one or more encryption keys.

The summary is intended to provide an overview of the subject matter described in this disclosure. It is not intended to provide an exclusive or exhaustive explanation of the systems, device, and methods described in detail within the accompanying drawings and description below. Further details of one or more examples of this disclosure are set forth in the accompanying drawings and in the description below. Other features, objects, and advantages will be apparent from the description and drawings, and from the claims.

### BRIEF DESCRIPTION OF DRAWINGS

FIGS. 1A and 1B illustrate the environment of an example medical device system in conjunction with a patient and a heart of the patient, in accordance with one or more techniques of this disclosure.

FIG. 2 is a block diagram illustrating an example configuration of components of an implantable medical device (IMD), in accordance with one or more techniques of this disclosure.

FIG. 3 is a block diagram illustrating an example configuration of components of an example external device, in accordance with one or more techniques of this disclosure.

FIG. 4 is a flow diagram illustrating an example operation in accordance with one or more techniques of this disclosure.

Like reference characters denote like elements throughout the description and figures.

### DETAILED DESCRIPTION

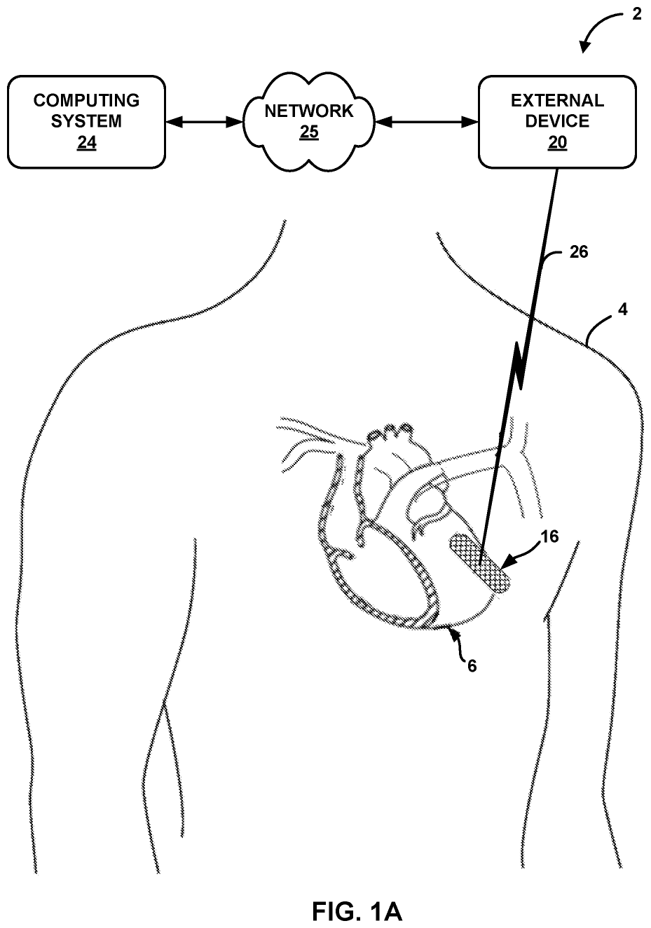

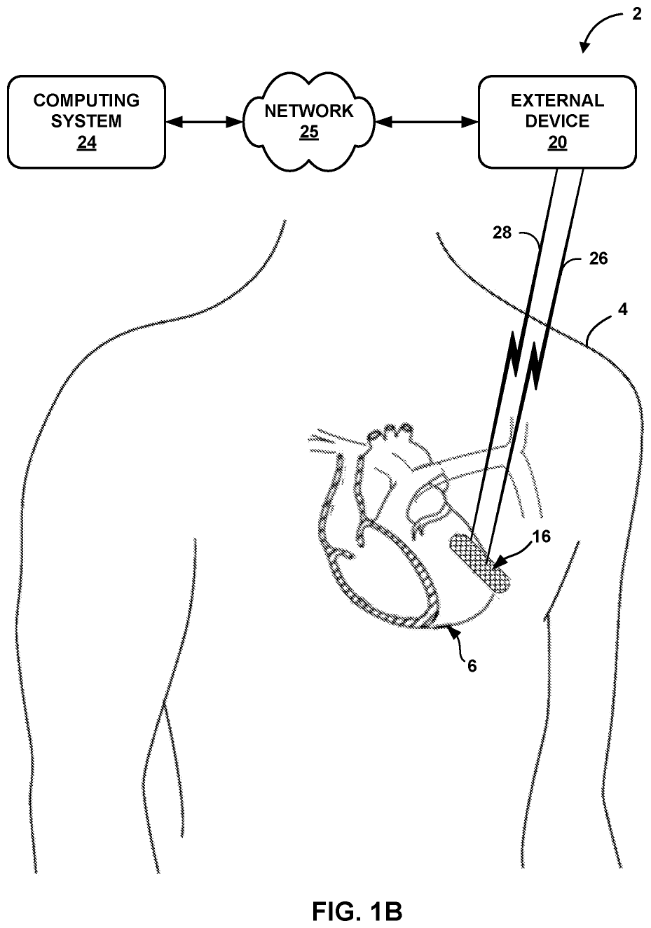

FIGS. 1A and 1B illustrate the environment of an example medical device system 2 in conjunction with a patient 4 and a heart 6 of patient 4, in accordance with one or more techniques of this disclosure. The example techniques may be used with an IMD 16, which may be in wireless communication with external device 20. External device 20 may also communicate with one or more external computing system(s), such as computing system 24, via network 25.

As shown in FIG. 1A, in some examples, IMD 16 is implanted in patient 4, such as implanted outside of a thoracic cavity of patient 4 (e.g., subcutaneously in the pectoral location illustrated in FIG. 1A). IMD 16 may be positioned near the sternum near or just below the level of heart 6, e.g., at least partially within the cardiac silhouette. In some examples, IMD 16 takes the form of a LINQ™ or LINQ II™ Insertable Cardiac Monitor (ICM), available from Medtronic, Inc., of Minneapolis, Minnesota.

Clinicians sometimes diagnose patients with cardiac conditions based on one or more observed physiological signals collected by physiological sensors, such as electrocardiogram (ECG) electrodes, electrogram (EGM) electrodes, chemical sensors, or temperature sensors. In some cases, clinicians apply non-invasive sensors to patients in order to sense one or more physiological signals while a patent is in a clinic for a medical appointment. However, in some examples, physiological markers (e.g., irregular heartbeats) of a cardiac condition are rare. As such, in these examples, a clinician may be unable to observe the physiological markers needed to diagnose a patient with a heart condition while monitoring one or more physiological signals of the patient during a medical appointment.

In some examples, IMD 16 includes a plurality of electrodes. The plurality of electrodes are configured to detect signals that enable processing circuitry of IMD 16 to determine current values of additional parameters associated with the cardiac and/or lung functions of patient 4. In some examples, the plurality of electrodes of IMD 16 are configured to detect a signal indicative of an electric potential of the tissue surrounding the IMD 16. Moreover, IMD 16 may additionally or alternatively include one or more accelerometers, temperature sensors, chemical sensors, light sensors, pressure sensors, in some examples.

External device 20 is configured to wirelessly communicate with IMD 16 as needed to provide or retrieve information. In some examples, external device 20 acts as an external programming device, e.g., medical device programmer, for IMD 16. External device 20 is an external computing device that a user, e.g., the clinician and/or patient 4, may use to communicate with IMD 16. For example, external device 20 may be a clinician programmer that the clinician uses to communicate with IMD 16 to retrieve information from IMD 16 and/or update one or more settings of IMD 16. Additionally, or alternatively, external device 20 may be a patient programmer that allows patient 4 to control certain operations of IMD 16 and/or view and modify one or more operational parameter values of IMD 16. The clinician programmer may include more programming features than the patient programmer. In other words, more complex or sensitive tasks may only be allowed by the clinician programmer to prevent an untrained patient from making undesired changes to IMD 16.

External device 20 may be a hand-held computing device with a display viewable by the user and an interface for providing input to external device 20 (i.e., a user input mechanism). For example, external device 20 may include a small display screen (e.g., a liquid crystal display (LCD) or a light emitting diode (LED) display) that presents information to the user. In addition, external device 20 may include a touch screen display, keypad, buttons, a peripheral pointing device, voice activation, or another input mechanism that allows the user to navigate through the user interface of external device 20 and provide input. If external device 20 includes buttons and a keypad, the buttons may be dedicated to performing a certain function, e.g., a power button, the buttons and the keypad may be soft keys that change in function depending upon the section of the user interface currently viewed by the user, or any combination thereof.

In other examples, external device 20 may be a larger workstation or a separate application within another multi-function device, rather than a dedicated computing device. For example, the multi-function device may be a notebook computer, tablet computer, workstation, one or more servers, cellular phone, personal digital assistant, or another computing device that may run an application that enables the computing device to operate as a secure device. In some examples, a wireless adapter coupled to the computing device enables external device 20 to establish a wireless communications link 26, such as a Bluetooth Low Energy connection, between the computing device and IMD 16.

When external device 20 is configured for use by the clinician, external device 20 may be used to transmit instructions to IMD 16. Example instructions may include requests to set electrode combinations for sensing and any other information that may be useful for programming into IMD 16. The clinician may also configure and store operational parameters for IMD 16 within IMD 16 with the aid of external device 20. In some examples, external device 20 assists the clinician in the configuration of IMD 16 by providing a system for identifying potentially beneficial operational parameter values.

Whether external device 20 is configured for clinician or patient use, external device 20 is configured to communicate with IMD 16 via wireless communication, such as via communication link 26. External device 20, for example, may communicate via near-field communication technologies (e.g., inductive coupling, NFC or other communication technologies operable at ranges less than 10-20 cm) and far-field communication technologies (e.g., RF telemetry according to the 802.11, Bluetooth, or Bluetooth Low Energy specification sets, or other communication technologies operable at ranges greater than near-field communication technologies).

External device 20 may also be configured to communicate with computing system 24 via network 25. Computing system 24 may comprise computing devices configured to allow a user to interact with IMD 16, or data collected from IMD 16, via network 25. For example, computing system 24 may include one or more handheld computing devices, computer workstations, servers or other networked computing devices. In some examples, computing system 24, network 25, and external device 20 may be implemented by the Medtronic Carelink™ Network or other patient monitoring system. In some examples, external device 20 may be configured to receive data from IMD 16, e.g., daily or otherwise according to a schedule, and transmit the data to computing system 24 via network 25.

Network 25 may include one or more computing devices (not shown), such as one or more non-edge switches, routers, hubs, gateways, security devices such as firewalls, intrusion detection, and/or intrusion prevention devices, servers, computer terminals, laptops, printers, databases, wireless mobile devices such as cellular phones or personal digital assistants, wireless access points, bridges, cable modems, application accelerators, or other network devices. Network 25 may include one or more networks administered by service providers, and may thus form part of a large-scale public network infrastructure, e.g., the Internet. Network 25 may provide computing devices, such as external device 20, computing system 24, and IMD 16, access to the Internet, and may provide a communication framework that allows the computing devices to communicate with one another. In some examples, network 25 may be a private network that provides a communication framework that allows computing system 24, IMD 16, and/or external device 20 to communicate with one another but isolates one or more of computing system 24, IMD 16, or external device 20 from devices external to network 25 for security purposes. In some examples, the communications between computing system 24, IMD 16, and external device 20 are encrypted.

In general, IMD 16 and external device 20 may exchange information using at least one communication protocol. Communication protocols define sets of rules that define one or more aspects of data exchange between two or more entities of a network. In some examples, communication protocols are stored as lists of computer-readable instructions and communication protocols may be executed by any combination of hardware (e.g., physical circuitry) and software. An organization, such as a medical device manufacturer, may create its own communication protocols, license communication protocols from a third party, use open source communication protocols, or perform any combination thereof. In some examples, a communication protocol includes security provisions, such as password requirements and data encryption in order to secure the transfer of data between two or more devices in a network.

Such information exchanged between IMD 16 and external device 20 may include information collected/sensed by IMD 16 and sent to external device 20, such as sensed physiological or biometric data from patient 4, diagnostic determinations made based on the sensed physiological or biometric data, therapy data associated with a therapy delivered to patient 4, performance data regarding operation and performance of IMD 16 (e.g., power level information, information regarding strengths of signals received, information regarding frequency of received interrogation requests, remaining battery life, etc.). The information may also include information sent by external device 20 to IMD 16, such as instructions, such as requests to set electrode combinations for sensing, and/or any other information (e.g., operational parameter values) that may be useful for programming into IMD 16.

IMD 16 and external device 20 may establish a wireless connection between IMD 16 and external device 20, such as in the form of communication link 26, in order to exchange information using at least one communication protocol. To establish a wireless connection between IMD 16 and external device 20, IMD 16 and external device 20 may perform a pairing process, during which IMD 16 and external device 20 may exchange information to form a trusted relationship prior to being able to communicate certain information with one another. In the example where IMD 16 and external device 20 attempt to establish a Bluetooth connection, IMD 16 and external device 20 may perform a pairing process according to Bluetooth specifications. Similarly, in the example where IMD 16 and external device 20 attempt to establish a Bluetooth Low Energy connection, IMD 16 and external device 20 may perform a pairing process according to Bluetooth Low Energy specifications.

As part of the pairing process or after successfully performing the pairing process, IMD 16 and external device 20 may establish a secure communications channel over communication link 26 in order to communicate one or more encryption keys between IMD 16 and external device 20. IMD 16 and external device 20 may use the one or more encryption keys to encrypt the information exchanged between IMD 16 and external device 20 via the link layer and/or application layer of communication link 26.

In some examples, IMD 16 and external device 20 may establish the secure communication channel over communication link 26 using a cryptographic protocol, such as Transport Layer Security (TLS). That is, in some examples, IMD 16 and external device 20 may establish a secure communication channel over communication link 26 in the form of a TLS tunnel. To establish a TLS tunnel, IMD 16 and external device 20 may perform a TLS handshake procedure, such as according to the specifications of TLS 1.3, to establish a secure TLS tunnel between IMD 16 and external device 20 by generating a session key associated with the TLS tunnel over communication link 26. IMD 16 and external device 20 may therefore use the session key to encrypt communications between IMD 16 and external device 20 in order to negotiate one or more encryption keys. The TLS tunnel may be opaque to a BLE connection and may therefore act as an out-of-band communication for the BLE connection.

IMD 16 and external device 20 may negotiate, via the secure tunnel, one or more encryption keys used to encrypt information exchanged between IMD 16 and external device 20 over communication link 26. The one or more encryption keys can be used to for link layer and/or application layer encryption between IMD 16 and external device 20. In some examples, IMD 16 may generate one or more encryption keys used for link layer and/or application layer encryption, encrypt the one or more encryption keys using the session key associated with the secure TLS tunnel, and send the one or more encryption keys to external device 20 via the secure TLS tunnel. In some examples, external device 20 may generate one or encryption keys used for link layer and/or application layer encryption, encrypt the one or more encryption keys using the session key associated with the secure TLS tunnel, and send the one or more encryption keys to IMD 16 via the secure TLS tunnel, and IMD 16 may receive the one or more encryption keys from external device 20 via the secure TLS tunnel.

IMD 16 may securely communicate with external device 20 using the one or more encryption keys. For example, IMD 16 may encrypt, using the one or more encryption keys, information sent via the link layer and/or application layer of communication link 26 to external device 20. Similarly, IMD 16 may decrypt, using the one or more encryption keys, encrypted information from external device 20 via the link layer and/or application layer of communication link 26.

In the example shown in FIG. 1B, while IMD 16 and external device 20 may communicate via communication link 26 to negotiate one or more encryption keys used to securely transfer information between IMD 16 and external device 20, IMD 16 and external device 20 may use a separate communication link, such as communication link 28, which is a wireless communication link different from communication link 26 to securely transfer information between IMD 16 and external device 20. Communication link 28 may be a wireless connection that uses a communication technique and/or communication protocol different from communication link 26. For example, while communication link 26 is a BLE communication link, communication link 28 may be an inductive coupling communication link (e.g., a communication link that implements a Tel B communication protocol).

IMD 16 may, in response to negotiating one or more encryption keys with external device 20 via communication link 28, establish communication link 28 with external device 20. For example, IMD 16 and external device 20 may perform any suitable pairing process or other process to establish communication link 28 between IMD 16 and external device 20 that is different from (e.g., uses a different communication protocol than) communication link 26.

IMD 16 may securely communicate with external device 20 using the one or more encryption keys over communication link 28. For example, IMD 16 may encrypt, using the one or more encryption keys, information that IMD 16 may send, via communication link 28, to external device 20. Similarly, external device 20 may encrypt, using the one or more encryption keys, information that external device 20 may send, via communication link 28, to IMD 16.

Although external device 20 is illustrated in FIGS. 1A and 1B as being a single device, in some examples (not illustrated), multiple external devices may perform the functions of external device 20.

Although in one example IMD 16 takes the form of an ICM, in other examples, IMD 16 takes the form of any combination of implantable cardioverter defibrillators (ICDs), pacemakers, cardiac resynchronization therapy devices (CRT-Ds), spinal cord stimulation (SCS) devices, deep brain stimulation (DBS) devices, left ventricular assist devices (LVADs), implantable sensors, orthopedic devices, or drug pumps, as examples. Moreover, techniques of this disclosure may be used to communicate with any one of the aforementioned IMDs. Moreover, techniques described in this disclosure may be applied to send and receive physiological data associated with patient 4 between two or more devices, where none of the two or more devices are implantable devices. Additionally, in some examples, techniques described in this disclosure may be applied to send and receive physiological data associated with patient 4 between two or more devices, where none of the two or more devices are medical devices.

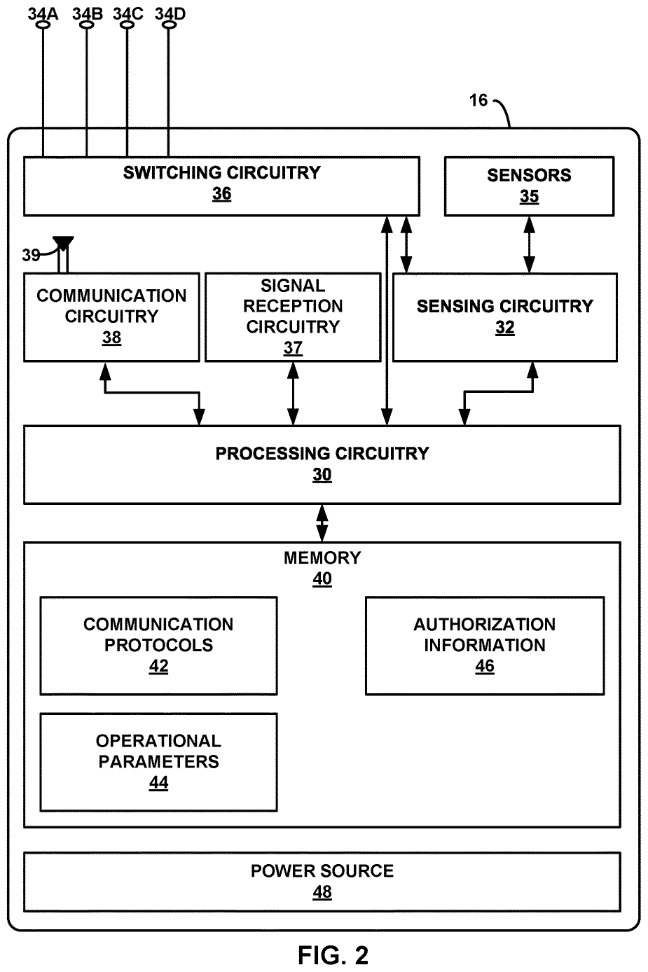

FIG. 2 is a block diagram illustrating an example configuration of components of IMD 16 in accordance with one or more techniques of this disclosure. In the example of FIG. 2, IMD 16 includes processing circuitry 30, sensing circuitry 32, electrodes 34A-34D (collectively, “electrodes 34”), sensors 35, switching circuitry 36, signal reception circuitry 37, communication circuitry 38, antenna 39, memory 40, and power source 48. Memory 40 is configured to store communication protocols 42, operational parameters 44, and/or collected physiological data.

Power source 48 is configured to deliver operating power to the components of IMD 16. Power source 48 may include a battery and a power generation circuit to produce the operating power. In some examples, the battery is rechargeable to allow extended operation. In some examples, recharging is accomplished through proximal inductive interaction between an external charger and an inductive charging coil within external device 18. Power source 48 may include any one or more of a plurality of different battery types, such as nickel cadmium batteries and lithium ion batteries.

Processing circuitry 30, in one example, may include one or more processors that are configured to implement functionality and/or process instructions for execution within IMD 16. For example, processing circuitry 30 may be capable of processing instructions stored in memory 40. Processing circuitry 30 may include, for example, microprocessors, digital signal processors (DSPs), application specific integrated circuits (ASICs), field-programmable gate arrays (FPGAs), or equivalent discrete or integrated logic circuitry, or a combination of any of the foregoing devices or circuitry. Accordingly, processing circuitry 30 may include any suitable structure, whether in hardware, software, firmware, or any combination thereof, to perform the functions ascribed herein to processing circuitry 30.

Sensing circuitry 32 monitors electrical cardiac signals from any combination of electrodes 34A-34D (collectively, “electrodes 34”). In some examples, sensing circuitry 32 may include one or more amplifiers, filters, and analog-to-digital converters. For example, sensing circuitry 32 may include one or more detection channels, each of which may include an amplifier. The detection channels may be used to sense cardiac signals, such as a cardiac EGM. Some detection channels may detect events, such as R-waves, P-waves, and T-waves and provide indications of the occurrences of such events to processing circuitry 30. Additionally, or alternatively, some channels may detect cardiac EGM signals from a particular combination of electrodes 34. One or more other detection channels may provide signals to an analog-to-digital converter, for conversion into a digital signal for processing, analysis, storage, or output by processing circuitry 30.

Each detection channel of sensing circuitry 32 may include a filter configured to pass a custom range of frequency values. For example, sensing circuitry 32 may include one or more narrow band channels, each of which may include a narrow band filtered sense-amplifier. Additionally, or alternatively, sensing circuitry 32 may include one or more wide band channels, each of which include an amplifier with a relatively wider pass band than the narrow band channels. Signals sensed by the narrow band channels and the wide band channels of sensing circuitry 32 may be converted to multi-bit digital signals by an analog-to-digital converter (ADC) provided by, for example, sensing circuitry 32 or processing circuitry 30. In some examples, processing circuitry 30 analyzes the digitized version of signals from sensing circuitry 32. In other examples, processing circuitry 30 stores the digitized versions of the signals in memory 40 and outputs the digitized versions of the signals via communication circuitry 38, or any combination thereof.

Processing circuitry 30 may use switching circuitry 36 to select, e.g., via a data/address bus, which of electrodes 34 to use for sensing cardiac signals. Switching circuitry 36 may include a switch array, switch matrix, multiplexer, or any other type of switching device suitable to selectively couple energy to selected electrodes.

In some examples, sensing circuitry 32 is electrically coupled to sensors 35. Sensors 35 may include any combination of accelerometers, temperature sensors, chemical sensors, light sensors, and pressure sensors. Sensors 35 may, for example, sense one or more physiological parameters indicative of a heart condition. Additionally, or alternatively, an accelerometer of sensors 35 may sense data indicative of at least one of patient posture and patient activity.

Signal reception circuitry 37 may include hardware, firmware, software or any combination thereof for receiving signals from another device, such as external device 20. Signal reception circuitry 37 may be powered by power source 48, “listening” for signals from external device 20. In other examples, power source 48 may power signal reception circuitry 37 every 250 milliseconds (ms) for a period of time, where the period of time lasts for greater than 0.1 ms and less than 50 ms. In this way, signal reception circuitry 37 may alternate between an “off” state and an “on” state, where signal reception circuitry 37 is configured to detect signals while signal reception circuitry 37 is being powered by power source 48 during the on state.

Communication circuitry 38 may include any suitable hardware, firmware, software or any combination thereof for communicating with another device, such as external device 20. Under the control of processing circuitry 30, communication circuitry 38 may receive downlink telemetry from, as well as send uplink telemetry to, external device 20 or another device with the aid of an internal or external antenna, e.g., antenna 39. In addition, processing circuitry 30 may communicate with a networked computing device via an external device (e.g., external device 20) and a computer network, such as the Medtronic CareLink® Network developed by Medtronic, Inc.

Communication circuitry 38 may include any combination of a radio (e.g., a Bluetooth® radio and/or a Bluetooth® Low Energy radio), magnetic induction circuitry (e.g., near-field magnetic induction communication circuitry), an electronic oscillator, frequency modulation circuitry, frequency demodulation circuitry, amplifier circuitry, and power switches such as a metal-oxide-semiconductor field-effect transistors (MOSFET), a bipolar junction transistor (BJT), an insulated-gate bipolar transistor (IGBT), a junction field effect transistor (JFET), or another element that uses voltage for its control. Signal reception circuitry 37 may, in some cases, be separate from communication circuitry 38. In other cases, signal reception circuitry 37 may be a component of, or a part of communication circuitry 38.

Memory 40 may be configured to store information within IMD 16 during operation. Memory 40 may include a computer-readable storage medium or computer-readable storage device. In some examples, memory 40 includes one or more of a short-term memory or a long-term memory. Memory 40 may include, for example, random access memories (RAM), dynamic random access memories (DRAM), static random access memories (SRAM), magnetic discs, optical discs, flash memories, or forms of electrically programmable memories (EPROM) or electrically erasable and programmable memories (EEPROM). In some examples, memory 40 is used to store data indicative of instructions for execution by processing circuitry 30.

In some examples, memory 40 is configured to store one or more communication protocols 42. Each protocol of communication protocols 42 may define a set of rules that govern one or more aspects of data exchange between IMD 16 and other devices (e.g., external device 20). In some examples, communication protocols 42 are stored as lists of computer-readable instructions and communication protocols may be executed by any combination of hardware (e.g., processing circuitry 30) and software. In some examples, communication protocols 42 includes a Bluetooth® protocol such as a Bluetooth Low Energy (BLE) protocol, a Session Initiation Protocol (SIP) based protocol, a Zigbee® protocol, a RF4CE protocol, a WirelessHART protocol, a 6LoWPAN (IPv6 over Low power Wireless Personal Area Networks) protocol, a Z-Wave protocol, an ANT protocol, an ultra-wideband (UWB) standard protocol, a radio frequency (RF) communication protocol, and/or other proprietary and non-proprietary communication protocols. In some examples, communication protocols 42 exclusively include the Bluetooth® protocol. Alternatively, in other examples, communication protocols 42 may include any combination of Bluetooth® protocols, protocols developed by the manufacturer of IMD 16, and protocols licensed from a third-party developer. For example, communication protocols 42 may include any combination of one or more Bluetooth® protocols and one or more other communication protocols, such as a communication protocol utilized for communications using magnetic induction.

In some examples, memory 40 is configured to store operational parameters 44. Operational parameters 44 may govern aspects of the operation of IMD 16. For example, operational parameters 44 may include combinations of electrodes 34 and sensors 35 for sensing physiological signals of patient 4. Additionally, or alternatively, operational parameters 44 may include a sampling rate for sampling analog signals sensed by electrodes 34 and sensors 35. Operational parameters 44 may be updated based on instructions received from an external device (e.g., external device 20) via communication circuitry 38. In some examples, processing circuitry 30 of IMD 16 updates operational parameters 44 only if instructions to update operational parameters 44 are received over a secure link.

IMD 16 may establish one or more communication links with another device, such as external device 20. For example, IMD 16 may receive data from external device 20 via communication circuitry 38, and IMD 16 may send data to external device 20 via communication circuitry 38. IMD 16 may send and receive data according to one or more of communication protocols 42. Communication protocols 42 may include one or more protocols and may enable IMD 16 to communicate according to a Bluetooth® protocol, such as a Bluetooth® Low Energy protocol, a magnetic induction communication protocol, and the like.

Processing circuitry 30 of IMD 16 is configured to periodically broadcast, via communication circuitry 38, advertisements (e.g., in the form of Bluetooth® advertising packets) that indicates IMD 16 is able to be paired with other external devices. External devices (e.g., external device 20) may be able to detect such advertisements and to establish one or more wireless communication links (e.g., communication link 26) with IMD 16 based on the information contained within the advertisements. For example, processing circuitry 30 may be configured to broadcast the advertisements every 30 seconds, every minute, and the like. Processing circuitry 30 may be configured to broadcast advertisements in accordance with one or more communication protocols 42, such as in accordance with a BLE communication protocol.

Processing circuitry 30 of IMD 16 may be configured to control the broadcasting of the advertisements. In some examples, processing circuitry 30 may be configured to control the broadcasting of advertisements based on receiving, such as via communication circuitry 38, a tissue conductance communication (TCC) sting signal, such as from another implantable medical device such as an implantable cardioverter defibrillator. For example, processing circuitry 30 may be configured to change the frequency of advertisement broadcasts (i.e., how often processing circuitry 30 broadcasts advertisements) in response to receiving, via communication circuitry 38, a TCC sting signal, such as increasing the frequency of advertisement broadcasts. In another example, instead of periodically broadcasting advertisements, processing circuitry 30 may be configured to broadcast one or more advertisements in response to receiving, via communication circuitry 38, a TCC sting signal.

In some examples, processing circuitry 30 may be configured to control the broadcasting of advertisements based on signals from one or more sensors 35 and/or and sensing circuitry 32. For example, accelerometer of sensors 35 may be configured to sense data indicative of at least one of patient body posture and patient activity, and processing circuitry 30 may be configured to control the broadcasting of advertisements based on the patient body posture and/or patient activity. For example, patient 4 may move to be in a particular posture, such as a sitting posture, to indicate that patient 4 or another entity (e.g., a clinician) would like to pair an external device (e.g., external device 20) with IMD 16. Processing circuitry 30 may therefore be configured to increase the frequency of advertisement broadcasts and/or to broadcast one or more advertisements in response to determining that patient 4 is in a particular body posture that indicates that an external device is to be paired with IMD 16.

In some examples, processing circuitry 30 may be configured to control the broadcasting of advertisements based on signals from one or more sensors 35 and/or and sensing circuitry 32 by using accelerometer of sensors 35 to detect the occurrence of a “tap” or another physical user interaction with IMD 16. For example, patient 4 or another user may “tap” or otherwise physically interact with IMD 16 to indicate that patient 4 or another entity (e.g., a clinician) would like to pair an external device (e.g., external device 20) with IMD 16. Processing circuitry 30 may therefore be configured to, in response to determining that the signals from one or more sensors 35 and/or and sensing circuitry 32 indicate the occurrence of a “tap” or another physical user interaction with IMD 16, increase the frequency of advertisement broadcasts and/or broadcast one or more advertisements.

Processing circuitry 30 may be configured to, in response to broadcasting one or more advertisements, receive, via communication circuitry 38, receive a connection request, such as a pairing request, from an external device, such as external device 20. Processing circuitry 30 may be configured to, in response to receiving a connection request from external device 20, perform a pairing process in accordance with one or more communication protocols 42, such as by performing a BLE pairing process.

As part of the pairing process or after performing the pairing process, processing circuitry 30 may be configured to verify whether external device 20 that sent the connection request is authorized to establish one or more communication links with IMD 16. For example, processing circuitry 30 may be configured to receive, from external device 20 and via communication circuitry 38, validation information that processing circuitry 30 may use to determine whether external device 20 is authorized to establish one or more communication links with IMD 16. Such validation information may be part of the connection request sent by external device 20 and received by IMD 16 or may be sent and received separately from the connection request.

Examples of validation information for determining whether external device 20 is authorized to establish one or more communication links with IMD 16 may include a specified code, a username and/or password, biometric information inputted at external device 20, information input to external device 20 via another device such as an RFID tag, and the like. Processing circuitry 30 may be configured to interpret the validation information to determine whether external device 20 is authorized to establish one or more communication links with IMD 16. For example, processing circuitry 30 may be configured to access authorization information 46 stored in memory 40 and/or compare the validation information with authorization information 46 to determine whether the validation information received by IMD 16 indicates that external device 20 is authorized to establish one or more communication links with IMD 16.

Processing circuitry 30 may be configured to, in response to successfully validating external device 20 as being authorized to establish one or more communication links with IMD 16, establish one or more communication links, such as communication link 26, with IMD 16. For example, if IMD 16 broadcasts advertisements in the form of BLE advertisements and, in response, receives a BLE connection request, processing circuitry 30 may be configured to, in response to successfully validating external device 20 as being authorized to establish a BLE communication links with IMD 16, establish a BLE communication link with IMD 16.

Processing circuitry 30 may therefore be configured to establish a secure communications channel over communication link 26 in order to communicate one or more encryption keys between IMD 16 and external device 20. IMD 16 and external device 20 may use the one or more encryption keys to encrypt the information exchanged between IMD 16 and external device 20 via the link layer and/or application layer of communication link 26.

In some examples, processing circuitry 30 is configured to establish a secure communication channel over communication link 26 using a cryptographic protocol, such as TLS. That is, in some examples, IMD 16 and external device 20 may establish a secure communication channel over communication link 26 in the form of a TLS tunnel. To establish a TLS tunnel, IMD 16 and external device may perform a TLS handshake procedure, such as according to the specifications of TLS 1.3, over communication link 26.

To perform a TLS 1.3 handshake, processing circuitry 30 may be configured to send, via communication circuitry 38 to external device 20, a Hello message, an indication of a list of supported cipher suites, and a key share. Processing circuitry 30 may be configured to, in response, receive, from external device 20 via communication circuitry 38, a key share of a chosen cipher suite out of the list of supported cipher suites, a digital certificate that identifies external device 20, and a time-stamped online certificate status protocol (OCSP) response signed by a certificate authority that indicates the authenticity of the digital certificate.

Because external device 20 performs OCSP stapling, external device 20 may bear the cost in providing the OCSP response by appending (i.e., stapling) the time-stamped OCSP response signed by a certificate authority in the response to the Hello message. By appending the time-stamped OCSP response signed by a certificate authority in the response, external device 20 may eliminate the need for IMD 16 to contact the certificate authority in order to authenticate the digital certificate.

Both the digital certificate and the time-stamped OCSP response may be encrypted using the key share received from external device 20. Processing circuitry 30 may be configured to decrypt the digital certificate and the OCSP response using the received key share and to verify, using the OCSP response, the authenticity of the digital certificate. Processing circuitry 30 may, in response to successfully verifying the authenticity of the digital certificate, generate a session key for encrypting communications between IMD 16 and external device 20 and send the session key to external device 20.

Processing circuitry 30 may therefore establish a secure tunnel in the form of a TLS tunnel by encrypting and decrypting communications between IMD 16 and external device 20 over communication link 26. Processing circuitry 30 may be configured to negotiate, via the secure tunnel, one or more encryption keys used to encrypt information exchanged between IMD 16 and external device 20 over communication link 26. The one or more encryption keys can be used to for link layer and/or application layer encryption between IMD 16 and external device 20. For example, IMD 16 and external device 20 may use the one or more encryption keys to encrypt and decrypt data packets sent and received via the link layer of communication link 26. In some examples, processing circuitry 30 may be configured to generate one or more encryption keys used for link layer and/or application layer encryption and may be configured to store the one or more encryption keys in memory 40 and to send the one or more encryption keys to external device 20 via the secure tunnel over communication link 26. That is, processing circuitry 30 may encrypt the one or more encryption keys using the session key associated with the secure tunnel and may send the encrypted one or more encryption keys over communication link 26 to external device 20.

In some examples, external device 20 may generate one or more encryption keys used for link layer and/or application layer encryption. External device 20 may encrypt the one or more encryption keys using the session key associated with the secure tunnel and may send the encrypted one or more encryption keys to IMD 16 via communication link 26. Processing circuitry 30 may be configured to receive, from external device 20 via communication circuitry 38 the encrypted one or more encryption keys over communication link 26. Processing circuitry 30 may therefore be configured to decrypt the encrypted one or more encryption keys and to store the one or more encryption keys in memory 40.

Processing circuitry 30 may be configured to store, in memory 40, the one or more encryption keys for securely communicating with external device 20. In addition, processing circuitry 30 may also be configured to store, in memory 40, an association between an identity of external device 20 and the one or more encryption keys for securely communicating with external device 20. For example, processing circuitry 30 may be configured to store, in memory 40, information identifying external device 20 (e.g., a unique identifier for external device 20) in such a way, such as in a defined data structure, such that the information identifying external device 20 is associated with the one or more encryption keys for securely communicating with external device 20 in memory 40.

IMD 16 may securely communicate with external device 20 using the one or more encryption keys. For example, processing circuitry 30 may be configured to encrypt, using the one or more encryption keys, information (e.g., data packets) that processing circuitry 30 may send via the link layer and/or application layer of communication link 26 to external device 20. Similarly, processing circuitry 30 may be configured to receive, via communication circuitry 38, encrypted information (e.g., data packets) over communication link 26 from external device 20, and processing circuitry 30 may be configured to use the one or more encryption keys to decrypt the encrypted information.

In some examples, while IMD 16 and external device 20 may communicate via communication link 26 to negotiate one or more encryption keys used to securely transfer information between IMD 16 and external device 20, IMD 16 and external device 20 may use a separate communication link, such as communication link 28, different from communication link 26 to securely transfer information between IMD 16 and external device 20. Communication link 28 may use a communication technique and/or communication protocol different from communication link 26. For example, while communication link 26 is a BLE communication link, communication link 28 may be an inductive coupling communication link.

Processing circuitry 30 may be configured to, in response to negotiating one or more encryption keys with external device 20, establish, via communication circuitry 38, communication link 28 with external device 20. For example, processing circuitry 30 may perform any suitable pairing process or other process to establish communication link 28 with external device 20 that is different from (e.g., uses a different communication protocol than) communication link 26.

IMD 16 may securely communicate with external device 20 using the one or more encryption keys over communication link 28. For example, processing circuitry 30 may be configured to encrypt, using the one or more encryption keys, information that processing circuitry 30 may send via communication link 28 to external device 20. Similarly, processing circuitry 30 may be configured to receive, via communication circuitry 38, encrypted information over communication link 28 from external device 20, and processing circuitry 30 may be configured to use the one or more encryption keys to decrypt the encrypted information.

In some examples, after the communication session between IMD 16 and external device 20 via communication link 26 has ended, IMD 16 may still be able to communicate sensitive information, such as protected health information, to external device 20, without reestablishing a communication link, such as a BLE connection, with external device 20. Instead, IMD 16 may be able to communicate with external device 20 by performing advertising. IMD 16 may perform advertising to broadcast information and/or to establish a connection with other devices (e.g., with external device 20). When IMD 16 performs advertising, IMD 16 may broadcast advertising packets that may include an advertising payload. In the example where IMD 16 broadcasts BLE advertising packets, each advertising packet may include a protocol data unit that includes a header and an advertising payload.

Devices that are within communications range of IMD 16 may be able to receive the advertising packets broadcasted by IMD 16 while IMD 16 performs advertising. To protect the sensitive information that may be carried by the advertising packets, processing circuitry 30 of IMD 16 may use the one or more encryption keys to encrypt data carried by advertising packets that are broadcast by IMD 16. For example, processing circuitry 30 may use the one or more encryption keys to encrypt the advertising payload of each of the advertising packets broadcasted by IMD 16. Encrypting data carried by the advertising packets may enable IMD 16 to securely transmit sensitive information, such as protected health information, in the advertising packets broadcasted by IMD 16.

For example, processing circuitry 30 may include, in the advertising payloads of advertising packets, protected health information associated with IMD 16 and/or patient 4. Such protected health information may include sensed physiological or biometric data from a patient (e.g., patient 4), diagnostic determinations made based on the sensed physiological or biometric data, therapy data associated with a therapy delivered to the patient, performance data regarding operation and performance of IMD 16 (e.g., power level information, information regarding strengths of signals received, information regarding frequency of received interrogation requests, remaining battery life, etc.), physiological data or biometric data of a patient, and/or information regarding therapy that was provided by IMD 16 to a patient 4.

IMD 16 may use one or more encryption keys negotiated by IMD 16 and external device 20 during a previous communication session to encrypt the advertising payloads of advertising packets. That is, IMD 16 may use one or more encryption keys that were negotiated via communicating with external device 20 via the TLS tunnel, as described above, and stored in memory 40, to encrypt the advertising payloads of advertising packets. As such, even though other devices within communications range of IMD 16 may be able to receive the advertising packets being broadcast by IMD 16, only external device 20 may be able to decrypt the encrypted advertising payloads of the advertising packets being broadcasted by IMD 16.

In some examples, IMD 16 may use the same one or more encryption keys used to securely communicate with external device 20 to encrypt the advertising payloads of advertising packets. That is, IMD 16 may use the same one or more encryption keys used to encrypt and decrypt information exchanged between IMD 16 and external device 20 over communication link 26 to encrypt the advertising payloads of advertising packets.

In some examples, IMD 16 may use one or more encryption keys to encrypt the advertising payloads of advertising packets that are different from the one or more encryption keys used to encrypt and decrypt information exchanged between IMD 16 and external device 20 over communication link 26. For example, when IMD 16 and external device 20 negotiates one or more encryption keys over the TLS tunnel, the one or more encryption keys may include a first one or more encryption keys and a second one or more encryption keys that IMD 16 may store in memory 40. The first one or more encryption keys may be used to encrypt and decrypt information exchanged between IMD 16 and external device 20 over communication link 26. The second one or more encryption keys may be used by IMD 16 to encrypt the advertising payloads of advertising packets that are broadcasted by IMD 16, and may be used by external device 20 to decrypt the advertising payloads of advertising packets broadcasted by IMD 16.

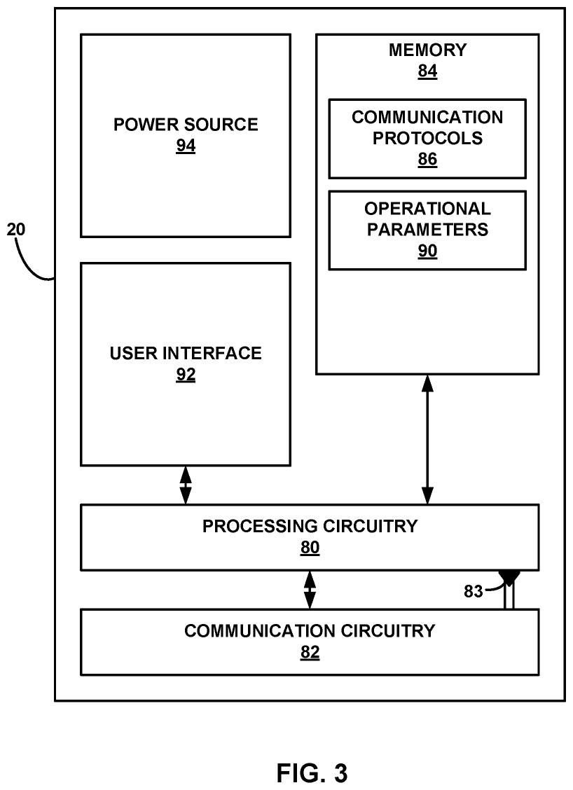

FIG. 3 is a block diagram illustrating an example configuration of components of external device 20 in accordance with one or more techniques of this disclosure. In the example of FIG. 3, external device 20 includes processing circuitry 80, communication circuitry 82, antenna 83, memory 84, user interface 92, and power source 94. Memory 84 is configured to store communication protocols 86 and operational parameters 90.

Processing circuitry 80, in one example, may include one or more processors that are configured to implement functionality and/or process instructions for execution within external device 20. For example, processing circuitry 80 may be capable of processing instructions stored in memory 84. Processing circuitry 80 may include, for example, microprocessors, DSPs, ASICs, FPGAs, or equivalent discrete or integrated logic circuitry, or a combination of any of the foregoing devices or circuitry. Accordingly, processing circuitry 80 may include any suitable structure, whether in hardware, software, firmware, or any combination thereof, to perform the functions ascribed herein to processing circuitry 80.

Communication circuitry 82 may include any suitable hardware, firmware, software or any combination thereof for communicating with another device, such as IMD 16. Under the control of processing circuitry 80, communication circuitry 82 may receive uplink telemetry from, as well as send downlink telemetry to, IMD 16 or another device with the aid of an internal or external antenna, e.g., antenna 83. In some examples, communication circuitry 82 includes a first set of communication circuitry configured for transmitting and receiving signals according to a communication protocol developed by the manufacturer of IMD 16 or a third-party developer. In some such examples, communication circuitry 82 further includes a second set of communication circuitry which defines a Bluetooth radio configured for transmitting and receiving signals according to Bluetooth communication protocols, including Bluetooth Low Energy protocols. However, communication circuitry 82 does not necessarily include separate sets of circuitry corresponding to different communication protocols. In some examples, communication circuitry 82 includes a single set of circuitry configured for transmitting and receiving signals according to a plurality of communication protocols.

In some examples, communication circuitry 82 includes any combination of a Bluetooth radio, an electronic oscillator, frequency modulation circuitry, frequency demodulation circuitry, amplifier circuitry, and power switches such as a MOSFET, a BJT, an IGBT, a JFET, or another element that uses voltage for its control.

Memory 84 may be configured to store information within external device 20 during operation. Memory 84 may include a computer-readable storage medium or computer-readable storage device. In some examples, memory 84 includes one or more of a short-term memory or a long-term memory. Memory 84 may include, for example, RAM, DRAM, SRAM, magnetic discs, optical discs, flash memories, or forms of EPROM or EEPROM. In some examples, memory 84 is used to store data indicative of instructions for execution by processing circuitry 80. Memory 84 may be used by software or applications running on external device 20 to temporarily store information during program execution.

External device 20 may exchange information with other devices via communication circuitry 82 according to one or more communication protocols 86. Communication protocols 86, stored in memory 84, may include sets of computer-readable instructions that determine how data is transmitted and processed.

Communication protocols 86 may include one or more communication protocols that are additionally included in communication protocols 42. In other words, IMD 16, and external device 20 may be configured to exchange information according to at least one common communication protocol. In some examples, the one or more common communication protocols include at least one Bluetooth communication protocol. Additionally, or alternatively, communication protocols 86 may include a set of communication protocols that are not available to IMD 16. In some examples, external device 20 is a consumer electronics device, such as a smartphone, a tablet, or a laptop computer. In some such examples, external device 20 may not be configured with communication protocols developed by the manufacturer of IMD 16.

Data exchanged between external device 20 and IMD 16 may include information collected/sensed by IMD 16 and sent to external device 20, such as sensed physiological or biometric data from a patient (e.g., patient 4), diagnostic determinations made based on the sensed physiological or biometric data, therapy data associated with a therapy delivered to the patient, performance data regarding operation and performance of IMD 16 (e.g., power level information, information regarding strengths of signals received, information regarding frequency of received interrogation requests, remaining battery life, etc.), physiological data or biometric data of a patient, and/or information regarding therapy that was provided by IMD 16 to a patient 4. Data exchanged between external device 20 and IMD 16 may also include any of operational parameters 90 stored in memory 84. External device 20 may transmit data including computer readable instructions which, when implemented by IMD 16, may control IMD 16 to change one or more operational parameters 90 according to operational parameters 90 and/or export collected data. For example, processing circuitry 80 may export instructions to IMD 16 requesting IMD 16 to update electrode combinations for stimulation or sensing according to operational parameters 90.

Processing circuitry 80 may be configured to receive, via communication circuitry 82, advertisements, such as BLE advertisements, broadcasted by IMD 16 and may be configured to, in response, initiate a pairing process with IMD 16 to establish communication link 26. Processing circuitry 80 may be configured to perform such a pairing process in accordance with a communication protocol, such as BLE. For example, as part of performing the pairing process with IMD 16, processing circuitry 80 may be configured to wirelessly send, via communication circuitry 82, a connection request, such as a pairing request, to IMD 16 based on the information included in the advertisements broadcasted by IMD 16.

As part of the pairing process or after performing the pairing process with IMD 16, processing circuitry 80 may be configured to send, via communication circuitry 82, validation information that IMD 16 may use to determine whether external device 20 is authorized to establish one or more communication links with IMD 16. Such validation information may be part of the connection request sent by external device 20 and received by IMD 16 or may be sent and received separately from the connection request. Examples of validation information for determining whether external device 20 is authorized to establish one or more communication links with IMD 16 may include a specified code, a username and/or password, biometric information inputted at external device 20, information input to external device 20 via another device such as an RFID tag, and the like.

In response to IMD 16 successfully validating external device 20 as being authorized to establish one or more communication links with IMD 16, external device 20 and IMD 16 may establish communication link 26 with IMD 16. Processing circuitry 80 may therefore be configured to establish a secure communication with IMD 16 over communication link 26 in order to communicate one or more encryption keys between IMD 16 and external device 20. IMD 16 and external device 20 may use the one or more encryption keys to encrypt the information exchanged between IMD 16 and external device 20 via the link layer and/or application layer of communication link 26.

In some examples, processing circuitry 80 is configured to establish a secure communication channel with IMD 16 over communication link 26 using a cryptographic protocol, such as TLS. That is, in some examples, IMD 16 and external device 20 may establish a secure communication channel over communication link 26 in the form of a TLS tunnel. To establish a TLS tunnel, IMD 16 and external device 12 may perform a TLS handshake procedure, such as according to the specifications of TLS 1.3, over communication link 26.

To perform a TLS 1.3 handshake, processing circuitry 80 may be configured to receive, via communication circuitry 82 from IMD 16, a Hello message, an indication of a list of supported cipher suites, and a key share. Processing circuitry 80 may be configured to, in response, send a key share of a chosen cipher suite out of the list of supported cipher suites, a digital certificate that identifies external device 20, and a time-stamped online certificate status protocol (OCSP) response signed by a certificate authority that indicates the authenticity of the digital certificate to IMD 16 via communication circuitry 82.

External device 20 may communicate with a certificate authority to receive the time-stamped OCSP response. Because external device 20 performs OCSP stapling, external device 20 may bear the cost in providing the OCSP response by appending (i.e., stapling) the time-stamped OCSP response signed by a certificate authority in the response to the Hello message. By appending the time-stamped OCSP response signed by a certificate authority in the response, external device 20 may eliminate the need for IMD 16 to contact the certificate authority in order to authenticate the digital certificate.

Both the digital certificate and the time-stamped OCSP response may be encrypted using the key share sent by external device 20. IMD 16 may, in response to successfully verifying the authenticity of the digital certificate, generate a session key for encrypting communications between IMD 16 and external device 20 and may send the session key to external device 20. Processing circuitry 80 may therefore be configured to receive, via communication circuitry 82, the session key from IMD 16.

Processing circuitry 80 may therefore establish a secure tunnel in the form of a TLS tunnel with IMD 16 by encrypting and decrypting communications between IMD 16 and external device 20 over communication link 26. Processing circuitry 80 may be configured to negotiate, via the secure tunnel, one or more encryption keys used to encrypt information exchanged between IMD 16 and external device 20 over communication link 26. The one or more encryption keys can be used to for link layer and/or application layer encryption between IMD 16 and external device 20. In some examples, processing circuitry 80 may be configured to generate one or more encryption keys used for link layer and/or application layer encryption and may be configured to store the one or more encryption keys in memory 84 and to send the one or more encryption keys to IMD 16 via the secure tunnel over communication link 26. That is, processing circuitry 80 may encrypt the one or more encryption keys using the session key associated with the secure tunnel and may send the encrypted one or more encryption keys over communication link 26 to IMD 16.

In some examples, IMD 16 may generate one or more encryption keys used for link layer and/or application layer encryption. IMD 16 may encrypt the one or more encryption keys using the session key associated with the secure tunnel and may send the encrypted one or more encryption keys to external device 20 via communication link 26. Processing circuitry 80 may be configured to receive, from IMD 16, the encrypted one or more encryption keys over communication link 26. Processing circuitry 80 may therefore be configured to decrypt the encrypted one or more encryption keys and to store the one or more encryption keys in memory 84.

Processing circuitry 80 may be configured to store, in memory 84, the one or more encryption keys for securely communicating with IMD 16. In addition, processing circuitry 30 may also be configured to store, in memory 84, an association between an identity of IMD 16 and the one or more encryption keys for securely communicating with IMD 16. For example, processing circuitry 80 may be configured to store, in memory 84, information identifying IMD 16 (e.g., a unique identifier for IMD 16) in such a way, such as in a defined data structure, such that the information identifying IMD 16 is associated with the one or more encryption keys for securely communicating with IMD 16 in memory 84.

External device 20 may securely communicate with IMD 16 using the one or more encryption keys. For example, processing circuitry 80 may be configured to encrypt, using the one or more encryption keys, information that processing circuitry 80 may send via the link layer and/or application layer of communication link 26 to IMD 16. Similarly, processing circuitry 80 may be configured to receive, via communication circuitry 82, encrypted information over communication link 26 from IMD 16, and processing circuitry 80 may be configured to use the one or more encryption keys to decrypt the encrypted information.

In some examples, while IMD 16 and external device 20 may communicate via communication link 26 to negotiate one or more encryption keys used to securely transfer information between IMD 16 and external device 20, IMD 16 and external device 20 may use a separate communication link, such as communication link 28, different from communication link 26 to securely transfer information between IMD 16 and external device 20. Communication link 28 may use a communication technique and/or communication protocol different from communication link 26. For example, while communication link 26 is a BLE communication link, communication link 28 may be an inductive coupling communication link.

Processing circuitry 80 may be configured to, in response to negotiating one or more encryption keys with IMD 16, establish, via communication circuitry 82, communication link 28 with IMD 16. For example, processing circuitry 80 may perform any suitable pairing process or other process to establish communication link 28 with IMD 16 that is different from (e.g., uses a different communication protocol than) communication link 26.

External device 20 may securely communicate with IMD 16 using the one or more encryption keys over communication link 28. For example, processing circuitry 80 may be configured to encrypt, using the one or more encryption keys, information that processing circuitry 80 may send via communication link 28 to IMD 16. Similarly, processing circuitry 80 may be configured to receive, via communication circuitry 82, encrypted information over communication link 28 from IMD 16, and processing circuitry 80 may be configured to use the one or more encryption keys to decrypt the encrypted information.

In some examples, after external device 20 ends the communication session with IMD over communication link 28, IMD 16 may broadcast advertising packets that contain encrypted advertising payloads. External device 20 may use the one or more encryption keys negotiated with IMD 16 to decrypt advertising payloads of advertising packets broadcasted by IMD 16. In some examples, processing circuitry 80 may use the same one or more encryption keys used to encrypt and decrypt data between IMD 16 and external device 20 to decrypt the encrypted advertising payloads.

In some examples, the one or more encryption keys negotiated with IMD 16 may include a first one or more encryption keys and a second one or more encryption keys that external device may store in memory 80. The first one or more encryption keys may be used to encrypt and decrypt information exchanged between IMD 16 and external device 20 over communication link 26 and/or communication link 28. The second one or more encryption keys may be used by IMD 16 to encrypt the advertising payloads of advertising packets that are broadcasted by IMD 16, and may be used by external device 20 to decrypt the advertising payloads of advertising packets broadcasted by IMD 16.

A user, such as a clinician or patient 4, may interact with external device 20 through user interface 92. User interface 92 includes a display (not shown), such as an LCD or LED display or other type of screen, with which processing circuitry 80 may present information related to and/or received from IMD 16 (e.g., EGM signals obtained from at least one electrode or at least one electrode combination). In addition, user interface 92 may include an input mechanism to receive input from the user. The input mechanisms may include, for example, any one or more of buttons, a keypad (e.g., an alphanumeric keypad), a peripheral pointing device, a touch screen, or another input mechanism that allows the user to navigate through user interfaces presented by processing circuitry 80 of external device 20 and provide input. In other examples, user interface 92 also includes audio circuitry for providing audible notifications, instructions or other sounds to patient 4, receiving voice commands from patient 4, or both. Memory 84 may include instructions for operating user interface 92 and for managing power source 94.

Power source 94 is configured to deliver operating power to the components of external device 20. Power source 94 may include a battery and a power generation circuit to produce the operating power. In some examples, the battery is rechargeable to allow extended operation. Recharging may be accomplished by electrically coupling power source 94 to a cradle or plug that is connected to an alternating current (AC) outlet. In addition, recharging may be accomplished through proximal inductive interaction between an external charger and an inductive charging coil within external device 20. In other examples, traditional batteries (e.g., nickel cadmium or lithium ion batteries) may be used. In addition, external device 20 may be directly coupled to an alternating current outlet to operate.

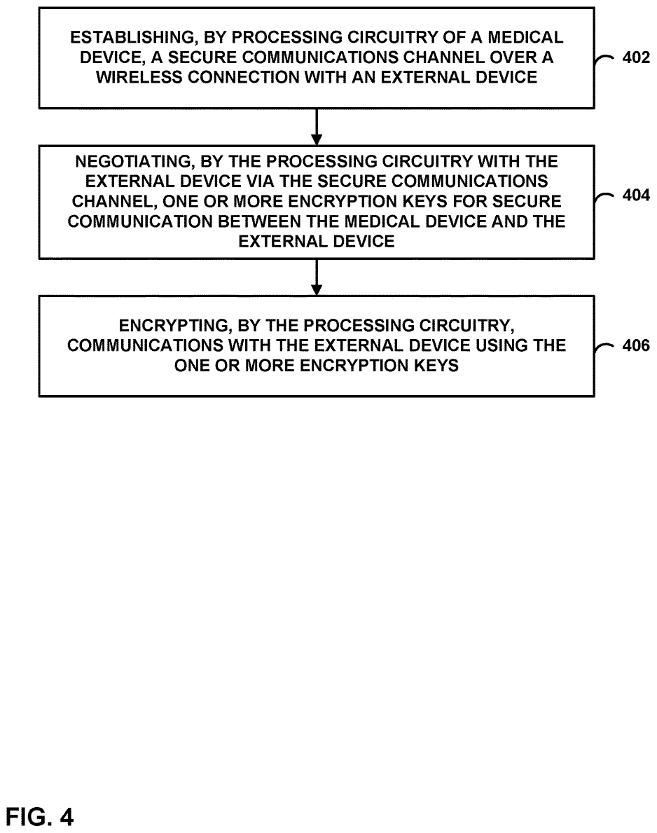

FIG. 4 is a flow diagram illustrating an example operation in accordance with one or more techniques of this disclosure. The example operation is described with respect to IMD 16 and external device 20 of FIGS. 1A, 1B, 2, and 3, and components thereof.

As shown in FIG. 4, processing circuitry 30 of IMD 16 may establish a secure communications channel over a wireless connection with an external device 20 (402). For example, processing circuitry 30 may establish a Transport Layer Security (TLS) tunnel over the wireless connection with the external device 20. In some examples, the wireless connection comprises a Bluetooth Low Energy (BLE) connection. In some examples, the medical device 16 comprises an implantable medical device (IMD).

In some examples, processing circuitry 30 may receive, via the wireless connection, validation information associated with the external device 20. Processing circuitry 30 may determine, based at least in part on the validation information, whether the external device 20 is authorized to establish a communication link with the medical device 16. Processing circuitry 30 may, in response to determining that the external device 20 is authorized to establish the communication link with the medical device 16, establish the secure communications channel over the wireless connection with the external device 20. In some examples, processing circuitry 30 may, in response to determining that the external device 20 is authorized to establish the communication link with the medical device 16, store, in the memory 40, an association between information identifying the external device 20 and the one or more encryption keys for secure communication between the medical device 16 and the external device 20.

Processing circuitry 30 of IMD 16 may negotiate, with the external device 20 via the secure communications channel, one or more encryption keys (404). In some examples, processing circuitry 30 may generate the one or more encryption keys and may send, via the secure communications channel to the external device 20, the one or more encryption keys. For example, processing circuitry 30 may encrypt the one or more encryption keys using a session key associated with the secure communications channel and may send, via the wireless connection to the external device, the encrypted one or more encryption keys.

In some examples, processing circuitry 30 may receive, via the secure communications channel and from the external device 20, the one or more encryption keys. Processing circuitry 30 may receive, via the wireless connection from the external device 20, the one or more encryption keys that are encrypted using a session key associated with the secure communications channel and may decrypt the one or more encryption keys using the session key associated with the secure communications channel.

Processing circuitry 30 of IMD 16 may encrypt communications with the external device 20 using the one or more encryption keys (406). In some examples, the communications include one or more of: sensed physiological data of a patient associated with the medical device, sensed biometric data of the patient, one or more diagnostic determinations made of the patient, data associated with a therapy delivered to the patient, performance data of the medical device, one or more instructions for the medical device, or one or more operational parameter values for the medical device. In some examples, processing circuitry 30 may encrypt the communications over at least one of: a link layer of the wireless connection or an application layer of the wireless connection with the external device 20 using the one or more encryption keys.

In some examples, to encrypt the communications with the external device 20 using the one or more encryption keys, processing circuitry 30 may encrypt communications via the wireless connection with the external device using the one or more encryption keys. In some examples, to encrypt the communications with the external device 20 using the one or more encryption keys, processing circuitry 30 may encrypt communications via a second wireless connection with the external device 20 using the one or more encryption keys.

In some examples, processing circuitry 30 may broadcast one or more advertisements that indicate the medical device 16 is able to be paired with other external devices. In some examples, processing circuitry 30 may determine that one or more signals of sensing circuitry 32 are indicative of an occurrence of a physical user interaction with the medical device 16 and may, in response to determining that the one or more signals are indicative of the occurrence of the physical user interaction, update how often the medical device 16 broadcasts the one or more advertisements. In some examples, processing circuitry 30 may receive a tissue conductance communication (TCC) sting signal and may, in response to receiving the TCC sting signal, update how often the medical device 16 broadcasts the one or more advertisements based at least in part on the TCC signal.

In some examples, processing circuitry 30 may use the one or more encryption keys to encrypt advertising payloads of advertising packets broadcasted by the medical device 16. In some examples, to negotiate the one or more encryption keys, processing circuitry 30 may negotiate, with the external device 20 via the secure communications channel, a plurality of encryption keys for secure communication between the medical device 16 and the external device 20. In some examples, to encrypt the communications with the external device 20 using the one or more encryption keys, processing circuitry 30 may encrypt communications with the external device 20 using a first one or more encryption keys of the plurality of encryption keys. In some examples, to encrypt the advertising payloads of advertising packets broadcasted by the medical device 16, processing circuitry 30 may encrypt, using a second one or more encryption keys of the plurality of encryption keys, the advertising payloads of the advertising packets broadcasted by the medical device 16.

Aspects of this disclosure include the following exemplary clauses.
Clause 1. A method comprising: establishing, by processing circuitry of a medical device, a secure communications channel over a wireless connection with an external device; negotiating, by the processing circuitry with the external device via the secure communications channel, one or more encryption keys for secure communication between the medical device and the external device; and encrypting, by the processing circuitry, communications with the external device using the one or more encryption keys.
Clause 2. The method of clause 1, wherein establishing the secure communications channel over the wireless connection with the external device further comprises: establishing, by the processing circuitry, a Transport Layer Security (TLS) tunnel over the wireless connection with the external device.
Clause 3. The method of any of clauses 1 and 2, wherein negotiating the one or more encryption keys further comprises: generating, by the processing circuitry, the one or more encryption keys; and sending, by the processing circuitry via the secure communications channel to the external device, the one or more encryption keys.
Clause 4. The method of clause 3, wherein sending, via the secure communications channel to the external device, the one or more encryption keys, further comprises: encrypting, by the processing circuitry, the one or more encryption keys using a session key associated with the secure communications channel; and sending, by the processing circuitry via the wireless connection to the external device, the encrypted one or more encryption keys.
Clause 5. The method of any of clauses 1-4, wherein negotiating the one or more encryption keys further comprises: receiving, by the processing circuitry via the secure communications channel and from the external device, the one or more encryption keys.
Clause 6. The method of clause 5, wherein receiving, via the secure communications channel and from the external device, the one or more encryption keys, further comprises: receiving, by the processing circuitry via the wireless connection from the external device, the one or more encryption keys that are encrypted using a session key associated with the secure communications channel; and decrypting, by the processing circuitry, the one or more encryption keys using the session key associated with the secure communications channel.
Clause 7. The method of any of clauses 1-6, further comprising: receiving, by the processing circuitry via the wireless connection, validation information associated with the external device; determining, by the processing circuitry and based at least in part on the validation information, whether the external device is authorized to establish a communication link with the medical device; and in response to determining that the external device is authorized to establish the communication link with the medical device, establishing, by the processing circuitry, the secure communications channel over the wireless connection with the external device.
Clause 8. The method of clause 7, further comprising: in response to determining that the external device is authorized to establish the communication link with the medical device, storing, by the processing circuitry in memory, an association between information identifying the external device and the one or more encryption keys for secure communication between the medical device and the external device.
Clause 9. The method of any of clauses 1-8, wherein encrypting the communications via the wireless connection with the external device using the one or more encryption keys further comprising: encrypting, by the processing circuitry, the communications over at least one of: a link layer of the wireless connection or an application layer of the wireless connection with the external device using the one or more encryption keys.
Clause 10. The method of any of clauses 1-9, further comprising: broadcasting, by the processing circuitry, one or more advertisements that indicate the medical device is able to be paired with other external devices.
Clause 11. The method of clause 10, further comprising: determining, by the processing circuitry, that one or more signals of sensing circuitry are indicative of an occurrence of a physical user interaction with the medical device; and in response to determining that the one or more signals are indicative of the occurrence of the physical user interaction, updating, by the processing circuitry, how often the medical device broadcasts the one or more advertisements.
Clause 12. The method of clause 10, further comprising: receiving, by the processing circuitry, a tissue conductance communication (TCC) sting signal; and in response to receiving the TCC sting signal, updating, by the processing circuitry, how often the medical device broadcasts the one or more advertisements based at least in part on the TCC signal.
Clause 13. The method of any of clauses 1-12, wherein encrypting the communications with the external device using the one or more encryption keys further comprises: encrypting, by the processing circuitry, communications via the wireless connection with the external device using the one or more encryption keys.
Clause 14. The method of any of clauses 1-12, wherein encrypting the communications with the external device using the one or more encryption keys further comprises: encrypting, by the processing circuitry, communications via a second wireless connection with the external device using the one or more encryption keys.
Clause 15. The method of clause 14, wherein the second wireless connection comprises an inductive coupling communication link.
Clause 16. The method of any of clauses 1-15, wherein the wireless connection comprises a Bluetooth Low Energy (BLE) connection.
Clause 17. The method of any of clauses 1-16, wherein the medical device comprises an implantable medical device (IMD).
Clause 18. The method of any of clauses 1-17, wherein the communications include one or more of: sensed physiological data of a patient associated with the medical device, sensed biometric data of the patient, one or more diagnostic determinations made of the patient, data associated with a therapy delivered to the patient, performance data of the medical device, one or more instructions for the medical device, or one or more operational parameter values for the medical device.
Clause 19. A medical device configured for wireless communication, wherein the medical device comprises: a memory; communication circuitry configured for wireless communication; and processing circuitry electrically coupled to the communication circuitry and the memory, wherein the processing circuitry is configured to: establish, a secure communications channel over a wireless connection via the communication circuitry with an external device; negotiate, with the external device via the secure communications channel, one or more encryption keys for secure communication between the medical device and the external device; and encrypt communications via the wireless connection with the external device using the one or more encryption keys.
Clause 20. The medical device of clause 19, wherein the processing circuitry configured to establish the secure communications channel over the wireless connection with the external device is further configured to: establish a Transport Layer Security (TLS) tunnel over the wireless connection with the external device.
Clause 21. The medical device of any of clauses 19 and 20, wherein to negotiate the one or more encryption keys, the processing circuitry is further configured to: generate the one or more encryption keys; and send, via the secure communications channel to the external device, the one or more encryption keys.
Clause 22. The medical device of clause 21, wherein to send, via the secure communications channel to the external device, the one or more encryption keys, the processing circuitry is further configured to: encrypt the one or more encryption keys using a session key associated with the secure communications channel; and send, via the wireless connection to the external device, the encrypted one or more encryption keys.
Clause 23. The medical device of any of clauses 19-22, wherein to negotiate the one or more encryption keys, the processing circuitry is further configured to: receive, via the secure communications channel and from the external device, the one or more encryption keys.
Clause 24. The medical device of clause 23, wherein to receive, via the secure communications channel and from the external device, the one or more encryption keys, the processing circuitry is further configured to: receive, via the wireless connection from the external device, the one or more encryption keys that are encrypted using a session key associated with the secure communications channel; and decrypt the one or more encryption keys using the session key associated with the secure communications channel.
Clause 25. The medical device of any of clauses 19-24, wherein the processing circuitry is further configured to: receive, via the wireless connection, validation information associated with the external device; determine, based at least in part on the validation information, whether the external device is authorized to establish a communication link with the medical device; and in response to determining that the external device is authorized to establish the communication link with the medical device, establish the secure communications channel over the wireless connection with the external device.
Clause 26. The medical device of clause 25, wherein the processing circuitry is further configured to: in response to determining that the external device is authorized to establish the communication link with the medical device, store, in the memory, an association between information identifying the external device and the one or more encryption keys for secure communication between the medical device and the external device.
Clause 27. The medical device of any of clauses 19-26, wherein to encrypt the communications via the wireless connection with the external device using the one or more encryption keys, the processing circuitry is further configured to: encrypt the communications over at least one of: a link layer of the wireless connection or an application layer of the wireless connection with the external device using the one or more encryption keys.
Clause 28. The medical device of any of clauses 19-27, wherein the processing circuitry is further configured to: broadcast one or more advertisements that indicate the medical device is able to be paired with other external devices.
Clause 29. The medical device of clause 28, wherein the processing circuitry is further configured to: determine that one or more signals of sensing circuitry are indicative of an occurrence of a physical user interaction with the medical device; and in response to determining that the one or more signals are indicative of the occurrence of the physical user interaction, update how often the medical device broadcasts the one or more advertisements.
Clause 30. The medical device of clause 28, wherein the processing circuitry is further configured to: receive a tissue conductance communication (TCC) sting signal; and in response to receiving the TCC sting signal, update how often the medical device broadcasts the one or more advertisements based at least in part on the TCC signal.
Clause 31. The medical device of any of clauses 19-30, wherein to encrypt the communications with the external device using the one or more encryption keys, the processing circuitry is further configured to: encrypt communications via the wireless connection with the external device using the one or more encryption keys.
Clause 32. The medical device of any of clauses 19-30, wherein to encrypt the communications with the external device using the one or more encryption keys, the processing circuitry is further configured to: encrypt communications via a second wireless connection with the external device using the one or more encryption keys.
Clause 33. The medical device of any of clauses 19-32, wherein the wireless connection comprises a Bluetooth Low Energy (BLE) connection.
Clause 34. The medical device of any of clauses 19-33, wherein the medical device comprises an implantable medical device (IMD).
Clause 35. The medical device of any of clauses 19-34, wherein the communications include one or more of: sensed physiological data of a patient associated with the medical device, sensed biometric data of the patient, one or more diagnostic determinations made of the patient, data associated with a therapy delivered to the patient, performance data of the medical device, one or more instructions for the medical device, or one or more operational parameter values for the medical device.
Clause 36. An apparatus comprising means for performing any of the methods of clauses 1-18.
Clause 37. A non-transitory computer-readable storage medium comprising program instructions that, when executed by processing circuitry of a medical device, cause the processing circuitry to perform the methods of any of clauses 1-18.

The techniques described in this disclosure may be implemented, at least in part, in hardware, software, firmware, or any combination thereof. For example, various aspects of the techniques may be implemented within one or more microprocessors, DSPs, ASICs, FPGAs, or any other equivalent integrated or discrete logic QRS circuitry, as well as any combinations of such components, embodied in external devices, such as physician or patient programmers, stimulators, or other devices. The terms “processor” and “processing circuitry” may generally refer to any of the foregoing logic circuitry, alone or in combination with other logic circuitry, or any other equivalent circuitry, and alone or in combination with other digital or analog circuitry.

For aspects implemented in software, at least some of the functionality ascribed to the systems and devices described in this disclosure may be embodied as instructions on a computer-readable storage medium such as RAM, DRAM, SRAM, magnetic discs, optical discs, flash memories, or forms of EPROM or EEPROM. The instructions may be executed to support one or more aspects of the functionality described in this disclosure.

In addition, in some aspects, the functionality described herein may be provided within dedicated hardware and/or software modules. Depiction of different features as modules or units is intended to highlight different functional aspects and does not necessarily imply that such modules or units must be realized by separate hardware or software components. Rather, functionality associated with one or more modules or units may be performed by separate hardware or software components, or integrated within common or separate hardware or software components. Also, the techniques could be fully implemented in one or more circuits or logic elements. The techniques of this disclosure may be implemented in a wide variety of devices or apparatuses, including an IMD, an external programmer, a combination of an IMD and external programmer, an integrated circuit (IC) or a set of ICs, and/or discrete electrical circuitry, residing in an IMD and/or external programmer.

1-15. (canceled)

16. A method comprising:
establishing, by processing circuitry of a medical device, a secure communications channel over a wireless connection with an external device;
negotiating, by the processing circuitry with the external device via the secure communications channel, one or more encryption keys for secure communication between the medical device and the external device; and
encrypting, by the processing circuitry, communications with the external device using the one or more encryption keys.

17. The method of claim 16, wherein establishing the secure communications channel over the wireless connection with the external device further comprises:
establishing, by the processing circuitry, a Transport Layer Security (TLS) tunnel over the wireless connection with the external device.

18. The method of claim 16, wherein negotiating the one or more encryption keys further comprises:
generating, by the processing circuitry, the one or more encryption keys;
encrypting, by the processing circuitry, the one or more encryption keys using a session key associated with the secure communications channel; and
sending, by the processing circuitry via the wireless connection to the external device, the encrypted one or more encryption keys.

19. The method of claim 16, wherein negotiating the one or more encryption keys further comprises:
receiving, by the processing circuitry via the wireless connection from the external device, the one or more encryption keys that are encrypted using a session key associated with the secure communications channel; and
decrypting, by the processing circuitry, the one or more encryption keys using the session key associated with the secure communications channel.

20. The method of claim 16, further comprising:
receiving, by the processing circuitry via the wireless connection, validation information associated with the external device;
determining, by the processing circuitry and based at least in part on the validation information, whether the external device is authorized to establish a communication link with the medical device; and
in response to determining that the external device is authorized to establish the communication link with the medical device, establishing, by the processing circuitry, the secure communications channel over the wireless connection with the external device.

21. The method of claim 16, wherein encrypting the communications via the wireless connection with the external device using the one or more encryption keys further comprises:
encrypting, by the processing circuitry, the communications over at least one of: a link layer of the wireless connection or an application layer of the wireless connection with the external device using the one or more encryption keys.

22. The method of claim 16, further comprising:
determining, by the processing circuitry, that one or more signals of sensing circuitry are indicative of an occurrence of a physical user interaction with the medical device; and
in response to determining that the one or more signals are indicative of the occurrence of the physical user interaction, updating, by the processing circuitry, how often the medical device broadcasts one or more advertisements that indicate the medical device is able to be paired with other external devices.

23. The method of claim 16, further comprising:
receiving, by the processing circuitry, a tissue conductance communication (TCC) sting signal; and
in response to receiving the TCC sting signal, updating, by the processing circuitry, how often the medical device broadcasts one or more advertisements that indicate the medical device is able to be paired with other external devices based at least in part on the TCC signal.

24. The method of claim 16, wherein encrypting the communications with the external device using the one or more encryption keys further comprises:
encrypting, by the processing circuitry, communications via a second wireless connection with the external device using the one or more encryption keys.

25. The method of claim 24, wherein the second wireless connection comprises an inductive coupling communication link.

26. The method of claim 16, further comprising:
encrypting, by the processing circuitry and using the one or more encryption keys, advertising payloads of advertising packets broadcasted by the medical device.

27. The method of claim 26,
wherein negotiating the one or more encryption keys further comprises negotiating, by the processing circuitry with the external device via the secure communications channel, a plurality of encryption keys for secure communication between the medical device and the external device,
wherein encrypting the communications with the external device using the one or more encryption keys further comprises encrypting, by the processing circuitry, communications with the external device using a first one or more encryption keys of the plurality of encryption keys, and
wherein encrypting the advertising payloads of advertising packets broadcasted by the medical device further comprises encrypting, by the processing circuitry and using a second one or more encryption keys of the plurality of encryption keys, the advertising payloads of the advertising packets broadcasted by the medical device.

28. The method of claim 16, wherein the wireless connection comprises a Bluetooth Low Energy (BLE) connection.

29. A medical device configured for wireless communication, wherein the medical device comprises:
a memory;
communication circuitry configured for wireless communication; and
processing circuitry electrically coupled to the communication circuitry and the memory, wherein the processing circuitry is configured to:
establish, via the communication circuitry, a secure communications channel over a wireless connection with an external device;
negotiate with the external device via the secure communications channel, one or more encryption keys for secure communication between the medical device and the external device; and
encrypt communications with the external device using the one or more encryption keys.

30. The medical device of claim 29, wherein to negotiate the one or more encryption keys, the processing circuitry is further configured to:
generate the one or more encryption keys;
encrypt the one or more encryption keys using a session key associated with the secure communications channel; and
send, via the wireless connection to the external device, the encrypted one or more encryption keys.

31. The medical device of claim 29, wherein to negotiate the one or more encryption keys, the processing circuitry is further configured to:
receive, via the wireless connection from the external device, the one or more encryption keys that are encrypted using a session key associated with the secure communications channel; and
decrypt the one or more encryption keys using the session key associated with the secure communications channel.

32. The medical device of claim 29, wherein the processing circuitry is further configured to:
receive, via the wireless connection, validation information associated with the external device;
determine, based at least in part on the validation information, whether the external device is authorized to establish a communication link with the medical device; and
in response to determining that the external device is authorized to establish the communication link with the medical device, establish the secure communications channel over the wireless connection with the external device.

33. The medical device of claim 29, wherein to encrypt the communications via the wireless connection with the external device using the one or more encryption keys, the processing circuitry is further configured to:
encrypt the communications over at least one of: a link layer of the wireless connection or an application layer of the wireless connection with the external device using the one or more encryption keys.

34. The medical device of claim 29, wherein the processing circuitry is further configured to:
determine that one or more signals of sensing circuitry are indicative of an occurrence of a physical user interaction with the medical device; and
in response to determining that the one or more signals are indicative of the occurrence of the physical user interaction, update how often the medical device broadcasts one or more advertisements that indicate the medical device is able to be paired with other external devices.

35. A non-transitory computer-readable storage medium comprising program instructions that, when executed by processing circuitry of a medical device, cause the processing circuitry to:
establish a secure communications channel over a wireless connection with an external device;
negotiate with the external device via the secure communications channel, one or more encryption keys for secure communication between the medical device and the external device; and
encrypt communications with the external device using the one or more encryption keys.
